(19) United States
(12) Patent Application Publication
(10) Pub. No.: US 2008/0114227 A1
(43) Pub. Date: May 15, 2008

(54) ANALYSIS DEVICE FOR IN VIVO DETERMINATION OF AN ANALYTE IN A PATIENT'S BODY

(75) Inventors: Hans-Peter Haar, Wiesloch (DE); Bernd Rosicke, Mannheim (DE)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: Roche Diagnostics Operations, Inc., Indianapolis, IN (US)

(21) Appl. No.: 11/937,872

(22) Filed: Nov. 9, 2007

(30) Foreign Application Priority Data
Nov. 15, 2006 (EP) 06023706.2

(51) Int. Cl. A61B 5/05

(52) U.S. Cl. 600/347; 600/345

(57) ABSTRACT

An analysis device for in vivo determination of an analyte in a patient's body, comprising a transdermal measurement probe (2) adapted to be introduced through the skin surface (4) into the body, with a probe head (6) including an electro-chemical analysis sensor (7) with two measurement electrodes (25, 26, 27), a body-wearable probe connection unit (3) adapted to be worn on the body and for connection to the transdermal measurement probe (2), a test circuit (22) connected with the measurement electrodes (25, 26, 27), wherein after contact of the measurement electrodes (25, 26, 27) with a body fluid the test circuit (22) generates a test signal characteristic for the desired analysis result, an evaluation circuit (23) for evaluating test signals from the test circuit (22) and for generating an information about the desired analysis result, wherein the test circuit (22) is integrated in the probe head (6) as part of probe head electronics (10), the probe head (6) is coupled to light conductor (8, 18), the probe head (6) includes an optical sensor (17) for converting electrical signals from the probe head electronics (10) into light signals, and for transmitting the light signals via the light conductor (8, 18) coupled to the probe head (6), and the probe connection unit (3) contains a light receiver (28) for receiving the light signals from the measurement probe (2) for further processing.

### CROSS REFERENCE TO RELATED APPLICATIONS

This application claims the benefit of European Patent Application No. 06023706.2 filed Nov. 15, 2006, which is hereby incorporated by reference in its entirety.

### BACKGROUND

The present invention relates to an analysis device for in vivo determination of an analyte in a patient's body, comprising a transdermal measurement probe with a probe head having an electro-chemical analysis sensor with two measurement electrodes, a body-wearable probe connection unit, a test circuit for connecting the measurement electrodes, and an evaluation unit. After contact of the measurement electrodes with body fluid the test circuit generates a test signal which is characteristic for the desired analysis result. In the evaluation circuit test signals of the test circuit are evaluated to obtain information about the desired analysis result.

Analysis systems for in vivo determination of an analyte in the body of a patient are specifically used when the analysis of a body fluid, particularly blood, needs to be conducted several times a day. A typical example is blood glucose monitoring in diabetics. If possible, the glucose level should be monitored continuously. The use of lancet devices to obtain a capillary blood sample is associated with pain and is accordingly time-consuming. Many patients consider this inconvenient which is the reason why they do not regularly determine the analyte in their bodies. In any case, the monitoring is conducted discontinuously, in practice no more than four to five times a day. Especially in diabetics, the fact that there is no continuous monitoring, and that the administration of insulin is inaccurate due to missing analysis results, can result in severe damages and late effects.

In the prior art, various analysis systems for continuous monitoring of analytes such as blood or lactate in blood or in (interstitial) body fluids are described. An analyte sensor based on electro-chemical measuring principles is introduced into the bloodstream or underneath the skin surface. One problem associated with these analysis systems is the generally insufficient reproducibility of the obtained analysis results. Often, these systems are relatively large and inflexible, and therefore accordingly inconvenient. The system hampers the mobility of the patient.

From U.S. Pat. No. 6,560,471 B1 an analysis system is known that involves arranging a part of an electro-chemical analysis sensor with a plurality of measurement electrodes underneath the skin surface. The analysis sensor portion positioned outside of the skin surface is coupled to a sensor controller containing a test circuit for evaluation of the test signals being determined by the measurement electrodes. The test signals being evaluated in the sensor controller are transmitted wirelessly to a display unit. The display unit is designed as an external device, or can be worn attached to the body or can be carried by the patients. In addition, the sensor controller can include displays for displaying information about the desired analytical result. Furthermore it is intended to produce a warning signal if, for example, a measured glucose value is lower than a predetermined value. In a display unit being designed as an external device, further evaluations and archiving of the measurements can be conducted, apart from the display of the analysis values.

The analysis device that has been presented by R. Beach, F. von Kuester, F. Moussy, in “Subminiature Implantable Potentiostat and modified commercial telemetry device for remote glucose monitoring” in IEEE Transactions on Instrumentation and Measurement, Vol. 48, no. 6, December 1999, uses a different approach. The analysis device comprises an electro-chemical glucose sensor, a test circuit designed as a potentiostat, an opto-coupling device for decoupling the test circuit from a wireless transmitter unit, and a power supply in the form of a battery. These components are implanted together underneath the patient's skin surface. The analysis device sends the measurement results corresponding to a glucose value as electro-magnetic waves to an external receiver which can comprise a computer. The analysis device is very large, with dimensions of 4 cm (length) by 2.6 cm (width) by 1.8 cm (height). Its implantation requires a complex surgical procedure. Furthermore, an additional surgical procedure is necessary whenever the battery of the analysis device is empty. The life of the battery has been indicated with one year at the most; however, under continuous use it is estimated to be only three months. Thus, the proposed device is unsuitable for daily use, and is associated with high costs for removal and implantation into the patient.

### SUMMARY OF THE INVENTION

On this basis, the invention addresses the problem of proposing an analysis device with an electro-chemical sensor for in vivo determination of an analyte in a patient's body with improved features compared to the analysis devices known from the art, especially regarding the comfort of wearing as well as measurement accuracy and reliability.

This problem is solved by an analysis device having the features according to claim 1.

The analysis device for in vivo determination of an analyte in a patient's body according to the invention comprises a transdermal measurement probe for introduction into the body through the skin surface. The probe has a probe head with an electro-chemical analysis sensor with at least two measurement electrodes and an optical transmitter to convert electrical signals from a probe head electronics into light signals, and to transmit the light signals through light conductors coupled to the probe head. A test circuit for connecting the measurement electrodes is integrated into the probe head as part of the probe head electronics. After contact of the measurement electrodes with bodily fluids the test circuit generates a test signal characteristic for the desired analysis result. A probe connection unit which can be worn attached to the body is used for connection to the transdermal measurement probe. The probe connection unit contains a light receiver to receive the light signals from the measurement probe for further processing. The analysis device further comprises an evaluation circuit for evaluating the test signals coming from the test circuit, and for obtaining information about the desired analysis result.

The analysis devices known from the art provide electrical wires between the measurement electrodes and the test circuit. Within the scope of the invention it was found that these electrical wires between the measurement electrodes and a test circuit positioned on the skin surface are not only perceived as inhibiting with respect to the mobility of the patient, but the use of these relatively long electrical wires can in fact lead to signal transfer problems. In the electro-chemical analysis sensors used, having two or even three measurement electrodes, currents in the range of pico-amperes are present. The impedance of the measurement assembly is in the range of several hundred megohms. This results in very high demands regarding the isolation of the signal wires and regarding the potential plug-in connections between the wires and the test circuit usually arranged outside of the body. Therefore, it is virtually impossible to exclude the occurrence of leakage currents that can bias the measurement results. These problems are even enhanced when a measurement assembly with three electrodes is used. Furthermore, due to the long wires, electro-magnetic currents can couple into the measurement assembly from the outside, generating noise voltages and noise currents, biasing the measurement results.

The present invention uses an entirely different approach to avoid long electric wires for transmitting test signals from the analysis sensor to the test circuit. The test circuit is integrated into the measurement probe as part of the probe head electronics. The electrical test signal produced by the test circuit is converted into light signals by an electro-optical converter arranged in the probe head. The light signals are then transmitted by light conductors from the measurement probe underneath the skin surface to the probe connection unit which can be worn outside the body attached at the body. Thus, the test signal is transmitted through the skin surface as a light signal being received in the probe connection unit by an optical light receiver and converted into an electrical signal in order to be further processed. Thus, the described problems in connection with wires are overcome. The optically transmitted signals are free from the above mentioned interferences. Especially, interferences from the outside that can influence long, parallel running electrical wires, and problems associated with the electrical connection of the wires with the test circuit and the probe connection unit, can be overcome.

In principle, there are two different procedures for transmitting data in the form of optical light signals from the measurement probe to the probe connection unit:
a) The test signals produced by the test circuit characteristic for the analysis result are transmitted through the light conductor as optical light signals. In this case, the “raw data” of the measurement assembly are transmitted. The transmitted data correspond to a current or resistance measurement value by means of which the analysis result can be determined in the evaluation unit.
b) The probe head electronics of the measurement probe preferably comprises the test circuit and also parts of the evaluation circuit. The test signal produced by the test circuit is transmitted to the evaluation circuit and is then further processed such that intermediate results, which can be converted into optical signals and transmitted to the probe connection unit are produced from the “raw data”. In the probe connection unit further part of the evaluation circuit is integrated in order to process the intermediate results for determining the analysis result.

The complete evaluation circuit can also be integrated in the probe head electronics.

In the probe connection unit the received light signal is re-converted into an electronic signal which is either displayed directly, or, for example, is transmitted wirelessly to an external display unit.

Advantageously, digital signals are transmitted by the light conductor. This involves integrating an analog-digital-converter (A/D-converter) into the probe head of the transdermal measurement probe converting the electrical signals of the probe head electronics into digital signals. In this manner, digital light signals can be transported from the measurement probe to the probe connection unit by the optical transmitter in the probe head. Transmitting of digital (optical) signals is far less sensitive for interferences than transmitting of analog optical signals. Processing in the probe connection unit is possible in a simple way known in the art.

The term “transdermal measurement probe” is meant in such manner that the measurement probe is inserted into the body through the skin surface. Thus, the probe is in the skin, i.e. in one of the plurality of skin (cutis) layers. Within the meaning of the invention measurement probes are also included that are inserted through the skin in such manner that they are arranged underneath the skin, i.e. subcutaneously. In any case, the transdermal measurement probe is arranged underneath the skin surface in the body.

Preferably, the test circuit is a potentiostat circuit, i.e. a circuit assembly with the function of a potentiostat, which is used to adjust the voltage between the two measurement electrodes (working electrode, counter electrode). For setting the voltage either a predetermined value can be used, or the value is adjusted.

Measurement probes with potentiostat circuits are particularly useful for long-term measurement of analytes in body fluids. Specifically, a continuous or quasi-continuous glucose measurement in the body is conducted. For long-term measurements, the electro-chemical analysis sensor arranged in the probe head of the transdermal measurement probe preferably has three measurement electrodes, i.e. a working electrode, a counter electrode and an additional reference electrode. This assembly enables a very exact measurement. Analogously to the chemical reaction, a very small electrical current is generated between the measurement electrodes. The current flows to the test circuit via the working electrode and the counter electrode. The accuracy of the measurement depends on exact monitoring of the voltage between the working electrode and the counter electrode in the measurement probe. In order to achieve this independently from interferences, the voltage between the working and a reference electrode is measured at high-impedance with the potentiostat circuit. This voltage is compared to a desired (predetermined) reference voltage between the working and the counter electrode. The potentiostat circuit adjusts the current through the counter electrode in such manner that the deviation of the measured voltage (actual value) between reference electrode and working electrode, and the voltage (reference value) between working electrode and counter electrode is eliminated, i.e. becomes zero. Thus, it is ensured that the desired potential is applied between the working electrode and the counter electrode. The current measured in the electric circuit, which flows through working electrode and counter electrode is a measure for the desired analysis result, and particularly, for glucose determination, a measure for the blood glucose content.

On the one hand, using the reference electrode create a very exact measurement. On the other hand, its high impedance of several 100 megohms results in the above described associated problems. Particularly in combination with the very small currents in the pico-ampere range the measurement result can be biased if the electrodes of the electro-chemical analysis sensor are positioned far away from the potentiostat circuit. Furthermore, biases can be generated by the electrical galvanic contacts, since parasitic surface resistances due to sweat can be occur between the non-isolated electrodes and the output or the supply connectors. According to the invention, both of these are avoided by integrating both, the electro-chemical analysis sensor and the test circuit designed as a potentiostat circuit, in the probe head of the transdermal measurement probe.

### BRIEF DESCRIPTION OF THE DRAWINGS

In the following, the invention is further explained by means of a preferred embodiment shown in the Figures. The features shown can be used individually or in combination to generate preferred embodiments of the invention.

FIG. 1 shows a schematic view of the analysis device with a transdermal measurement probe and a probe connection unit;

FIG. 2 shows a cross-sectional view of the transdermal measurement probe with a probe head and an light conductor;

FIG. 3 shows an enlarged a cross-sectional view of the probe head of FIG. 2;

FIG. 4 shows a cross-sectional view of the probe head on a second plain;

FIG. 5 shows the transdermal measurement probe of FIG. 2 with an introducer; and

FIG. 6 shows a detailed view of a portion of the light conductor with the introducer of FIG. 5.

### DETAILED DESCRIPTION

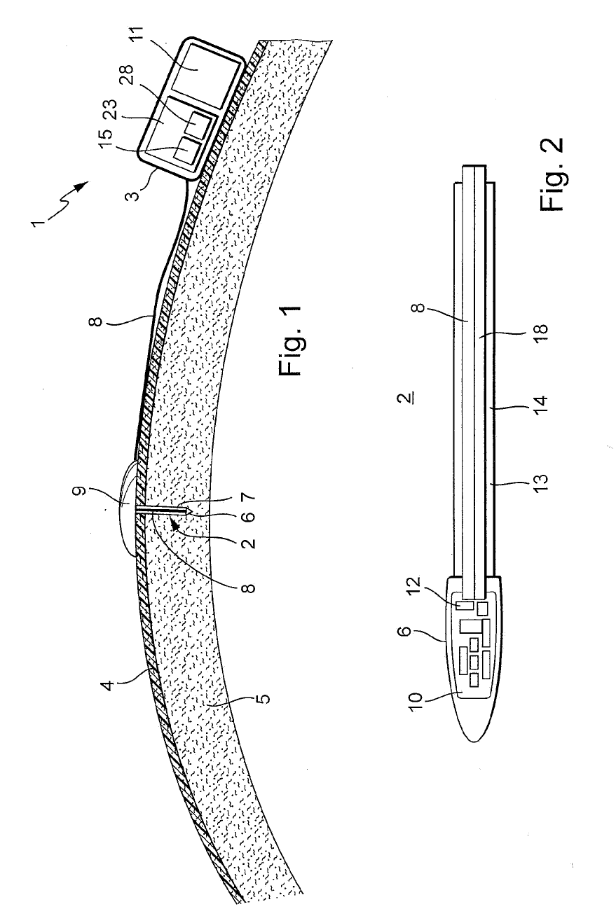

FIG. 1 shows an analysis device 1 according to the invention with a transdermal measurement probe 2 and a probe connection unit 3. Analysis device 1 is used to determine the glucose content of a patient in vivo. If, in the following, reference is made to the glucose determination, it is not intended to be a limitation of generality. Analysis device 1 is also suitable to determine other body fluid analytes which can be detected with an electro-chemical analysis sensor.

The transdermal measurement probe 2 is introduced through the skin surface 4 such that the measurement probe 2 is positioned in the skin 5 of a patient. The transdermal measurement probe 2 has a probe head 6 in which an electro-chemical analysis sensor 7 is integrated. Probe head 6 is connected with the probe connection unit 3 arranged on the skin surface 4 via a light conductor which is preferably designed as an optical fiber 8. A covering unit 9 arranged on the outside of the skin is used for stabilization of the transdermal measurement probe 2 in the skin 5. Preferably, it has the form of a mushroom head which increases the comfort of wearing. The covering unit 9 stabilizes the transdermal measurement probe 2 in the skin. This, on the one hand, prevents any unintended removal of the transdermal measurement probe 2 from the skin, and, on the other hand, excludes painful movements of the measurement probe 2 in the skin; i.e. the measurement probe 2 remains fixed in the skin.

Probe connection unit 3 comprises a power supply unit 11 to supply power to the transdermal measurement probe 2. In order to achieve this, an electro-optical converter 15 is provided to convert power into light which then is transported to the measurement probe via the optical fiber 8. An evaluation circuit 23 integrated in the probe connection unit 3 receives the test signals generated by the measurement probe 2, and evaluates them in order to create an analysis result. A light receiver 28 will convert the optically transmitted test signal into an electrical test signal which then can be processed by the evaluation circuit 23.

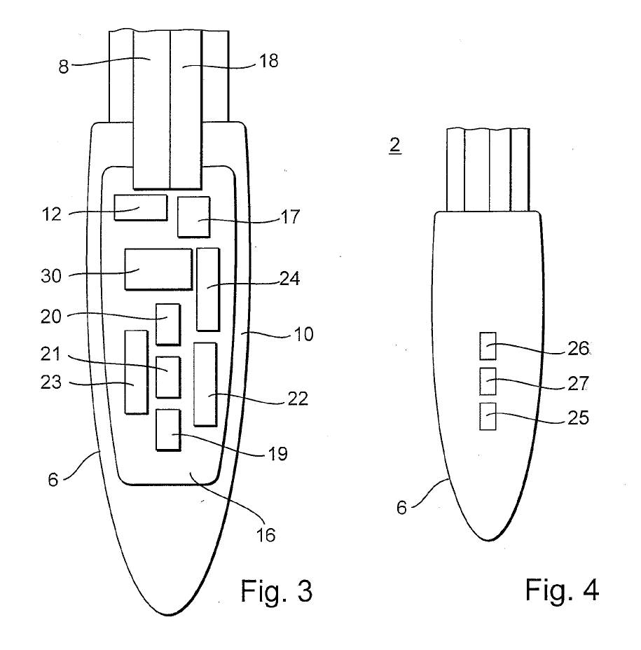

The optical fiber 8 connecting the measurement probe 2 with the probe connection unit 3 serves for data transfer of the test signals received by the analysis sensor 7 to the evaluation circuit 23 integrated in the probe connection unit. Furthermore, the optical fiber 8 also serves for transmitting power to the measurement probe 2. Measurement probe 2 comprises an optical receiver 12 (FIG. 3) which converts the received light back into electrical power, thus providing power to the probe head electronics 10 in the probe head 6.

In a particularly preferred embodiment, the opto-electrical converter of the power supply unit 11 is a laser diode 15 for transmitting power in the form of light to the measurement probe 2. A semiconductor laser or an alternating laser can also be used in the probe connection unit 3 instead of the laser diode 15. Preferably, an electro-optical laser is used allowing for a very efficient transmittance of power in the form of light. The use of VCSEL lasers has turned out to be especially suitable. These preferred lasers operate at a wavelength of 700 nm with an efficiency of 70%. The optical receiver 12 in the probe head 6 of the measurement probe 2 is then preferably designed as a photodiode adjusted to the VCSEL laser wavelength, with an efficiency of 40%.

FIG. 2 shows the transdermal measurement probe 2 with its probe head 6 and the optical fiber 8 coupled thereto. In a particularly preferred embodiment, additional to the optical fiber 8, another optical fiber 18 is coupled to the probe head 6. Preferably, one of the two optical fibers 8, 18 serves for power transfer to the measurement probe 2, whereas the other optical fiber 8, 18 is used for transfer of the optical data signals. In this manner, separation of the power transfer and the data transfer to the measurement probe 2 can be realized. Furthermore, it is also possible to use one of the optical fiber 8 for power transfer and at the same time for transfer of data signals from probe connection unit 3 to measurement probe 2, whereas the other optical fiber 18 is used for data transfer from the measurement probe 2 to the probe connection unit 3.

In an alternative embodiment with only one optical fiber 8, transfer from and to measurement probe 2 can occur in the form of light signals via the single optical fiber 8. Preferably, the light signals for data transfer are modulated in such a manner that power transfer and data transfer are simultaneously possible also with just one single optical fiber 8.

Since the transfer of the analysis values or measurements characterizing an analysis result takes place in the form of light, decoupling, and thus galvanic insulation, of the electro-chemical analysis unit and the electrical test circuit from the transfer is achieved. Furthermore, coupling of electrical interferences is prevented. Shielding measures can therefore be omitted. Particularly, by appropriate encapsulation of the measurement probe, galvanic coupling and thus the occurrence of residual current can be avoided.

The preferred embodiment shown in the Figures shows the measurement probe 2 with a canula 13 enclosing the optical fiber 8. Advantageously, the optical fiber 8 is at least enclosed in the area which is transdermally disposed in the skin 5.

The canula 13 does not necessarily have to be a rigid tube. Within the meaning of the present invention, the term “canula” also comprises net-like structures or tissue structures which also can be partially enhanced. Preferably, the canula 13 has a flexible sheath structure 14 with a high tensile strength. It is particularly preferably made of a fibrous tissue. Plastic or man-made fibers are particularly suitable for this purpose, with the use of synthetic fibers, e.g. specifically polyamide, being particularly preferred. However, other structures in the form of a canula are conceivable as well, provided they also exhibit the desired flexibility and tensile strength.

The employed light conductor 8, 18 preferably consist of a plastic material, i.e. they are “polymer light conductors” or “polymer optical fibers”. These materials are especially suitable for transfer of light, and they have little attenuation. Of course, glass fibers can be used as an alternative or other, preferably transparent materials, which are sufficiently suitable for transfer of light and for the intended use for connection to a transdermal measurement probe.

The light conductors 8, 18 preferably have a diameter of not more than 100 μm, preferably not more than 30 μm. The light conductors 8, 18 shown in FIG. 2 have a diameter of 10 μm which is considered as particularly preferred. This thin optical fiber allows for a high degree of formability, especially because it is very flexible and can be positioned very easily on the body. Due to its high flexibility, the user hardly notices the light conductors 8, 18 during daily use as they adjust to every movement of the body.

The relatively thin fibers with a diameter of approximately 10 μm furthermore have the advantage that the entire fiber, including the sheath structure 14, has a total diameter of less than 250 μm, preferably approximately 150 μm. As a result of this small diameter, the sensation of pain of the sensor in the skin is noticeably reduced. Due to the very thin polymer optical fiber, the sheath structure 14 can be designed such that is has a very high tensile strength. This ensures that the measurement probe keeps intact and will not be damaged, even when it is pulled out of the skin. The tissue of the sheath structure 14 itself is very smooth to ensure easy and pain-free sliding-out of the skin. Alternatively, the sheath structure can be designed to comprise a plurality of appropriately thin layers.

FIG. 3 shows a cross-section through the probe head 6 of the transdermal measurement probe 2, with the light conductor 8, 18 being coupled to the probe head 6. A circuit board 16 accommodating the probe head electronics 10 is provided in the probe head 6. Both light conductors 8, 18 are coupled to the probe head electronics in such manner that the optical receiver 12 being positioned at the end of the optical fiber 8, is adapted for converting the power transmitted in the form of light to electrical power to supply the probe head electronics 10. An optical transmitter 17 is positioned at the end of the second optical fiber 18. It converts electrical signals of the probe head electronics 10 into optical light signals and transmits them to the probe connection unit 3 via the optical fiber 18.

The probe head electronics 10 comprises three terminal areas 19, 20, 21 to attach the measurement electrodes 25, 26, 27 which are positioned on a plane above the cross-sectional plane shown in FIG. 3. This plane is shown in FIG. 4. A test circuit 22 is connected with the terminal areas 19, 20, 21 of the measurement electrodes of the analysis sensor 7 to generate a test signal after contact of the measurement electrodes 25, 26, 27 with a body fluid (interstitial fluid, blood) being characteristic for the desired glucose analysis result.

In the embodiment according to FIG. 3, the evaluation circuit 23 is integrated into the probe head electronics 10. The test signals generated by the test circuit 22 are evaluated in the evaluation circuit 23 to obtain information about the desired analysis result, in this case the patient's glucose content. An algorithm is implemented in evaluation unit 23 which processes deposited calibration data in form of a measurement curve or a table in such manner that a direct correlation between test signals and glucose content is given. The calibration data can also be modified externally, for example, modified calibration data can be supplied from the probe connection unit 3 via the optical fiber 8 into the evaluation circuit 23. By comparison of the test signals generated by the test circuit, the correlating glucose value will be determined. The determined information about the glucose content has the form of an electrical analog value being converted into a digital signal in an A/D-converter 24. The digital signal is then transmitted by optical transmitter 17 via the optical fiber 18 to the probe connection unit 3. Alternatively, it is also possible to convert the test signals generated by the test circuit 22, usually being analog electrical signals, in the A/D-converter 24 into digital values before they are transmitted to the evaluation circuit 23. The evaluation circuit 23 can be realized in the form of a software. Hereby, individual measurements can be temporarily stored, e.g. to avoid transfer conflicts or to implement real-time functions.

The measurement electrodes 25 to 27 are located above the terminal areas 19 to 21, as can be seen in FIG. 4. The measurement electrodes 25 to 27 are connected with the respective terminal areas 19 to 21. The working electrode 25 comprises an enzyme to enzymatically process the glucose molecules of the patient, and to measure the thereby produced electron as a current. For glucose determination a glucose oxidase is used as the enzyme. Furthermore, the working electrode 25 also contains manganese oxide. The measurement electrode 26 is called counter electrode, and it is made from platinum or gold. The measurement electrode 27 is the reference electrode, and is made from silver/silver chloride (Ag/AgCl).

The entire probe head 6 (including the light conductor) is hermetically sealed by an isolating protection sheath. The measurement probe 2 has openings in this external sheath, through which body fluids can enter and exit such that the body fluid can get in contact with the measurement electrodes 25 to 27. The openings located above the measurement electrodes 25 to 27 are closed with a cover film (not shown) which is formed from a biocompatible material. The cover film forms a permeable membrane through which a diffuse passage of the body fluid, particularly of interstitial fluid, is possible such that the fluid can reach the measurement electrodes 25 to 27.

The test circuit 22 is designed as a potentiostat circuit. This control circuit adjusts the voltage between the working electrode 25 and the counter electrode 26 to a predefined voltage value, depending on the working voltage measured between a reference electrode 27 and the working electrode 25. To perform long-term measurement of the blood glucose content, a voltage in the range of 200 mV (millivolts) and 300 mV (millivolts) must be permanently applied to the counter electrode 26. The hereto required power is in the range of nanowatt-seconds to microwatt-seconds (mWs-μWs). This requires permanent or at least intermittent power delivery to the transdermal measurement probe 2. Alternatively or in addition, a buffer battery 30 can be integrated in the probe head 6 to compensate for voltage fluctuations or power transfer fluctuations. This battery is very small, and can bridge the power supply only for a certain, relatively short period of time when no light is transmitted from the power supply unit 11 to the probe 2. As an alternative to the battery, another power storage unit, e.g. a capacitor, so called super-caps, or the like, can be used.

Altogether, the power consumption of the measurement probe 2 is rather small. If the probe head electronics 10 is implemented in an ASIC structure, the power consumption is approximately 1 μW (microwatt). If a VCSEL laser with an efficiency factor of 70% and an efficiency factor of 40% for accepting a wavelength-adjusted photodiode is used for power transmittance, the required energy demand is approx. 10 Joules per day. Thus, with a conventional AAA battery, the measurement probe 2 could be operated for about one month.

In an alternative embodiment of the transdermal measurement probe 2 a second electro-chemical analysis sensor can preferably be integrated into the probe head 6. This allows for designing a redundant measurement system to further increase measurement accuracy and precision. Alternatively, in addition to the glucose value, another analyte, for example lactate or the like, can be determined with the second electro-chemical analysis sensor. These double-sensor structures are also suitable either for improved quality assurance or for parallel measurement of a plurality of analytes in a body fluid, so a more comprehensive monitoring can be conducted. Also, in a further alternative embodiment, a plurality of electrodes or accessory electrodes can be disposed in the transdermal measurement probe 2 to carry out alternating current resistance measurements. In this case, four to five additional electrodes can advantageously be present. It is also possible to implement a thermometer in the measurement probe 2, in addition to the electro-chemical analysis sensor, to determine the temperature at the glucose measurement site. Temperature-dependant deviations can then be compensated, if necessary.

In another preferred embodiment, the measurement electrodes 25 to 27 are sterilized, particularly electron-beam sterilized. An embodiment is preferred in which the entire electro-chemical analysis sensor 7 is electron-beam sterilized. Especially preferably, the entire probe head 6 and/or the entire transdermal measurement probe 2, including the probe head electronics 10, are electron-beam sterilized. For this purpose, sterilization with electron-beams will be performed only after assembly of the measurement probe. Alternatively, a two-step production process can be performed in which the “sensor chemistry” (analysis sensor and measurement electrodes) is electron-beam sterilized, and is then, after having been protected against microbial re-contamination, mounted together with the (chemically) sterilized probe head electronics 10. Partly, a partial shielding of probe head electronics 10 during electron-beam (e-beam) treatment for sterilization is possible.

If not the entire, but only parts of the evaluation circuit 23 are integrated into probe head electronics 10, intermediate results will be determined from the test signals produced by the test circuit 22, which will be transmitted through light conductor 8, 18 to the probe connection unit 3. Probe connection unit 3 then comprises a further part of the evaluation circuit 23 to analyze and/or process the transmitted intermediate results. Furthermore, the probe connection unit 3 can also include a display for output of the analysis value, particularly the glucose content. Furthermore, a radio transmitter can be integrated into the probe connection unit 3 to transmit the values to an external unit. In the external unit, the analysis values can only be displayed, or they can also be processed and archived. Transmittance from the probe connection unit 3 to the external unit can take place over common radio transmission, for example by infrared or Bluetooth or wireless LAN connections.

Alternatively, part of the evaluation circuit 23 can also be integrated in the covering unit 9. Covering unit 9 can then also include a radio module to transmit the analysis results to an external unit. In this case, probe connection unit 3 only comprises power supply unit 11, including the required electro-optical converter. Furthermore, it can also be possible to integrate the entire probe connection unit 23 in the covering unit 9.

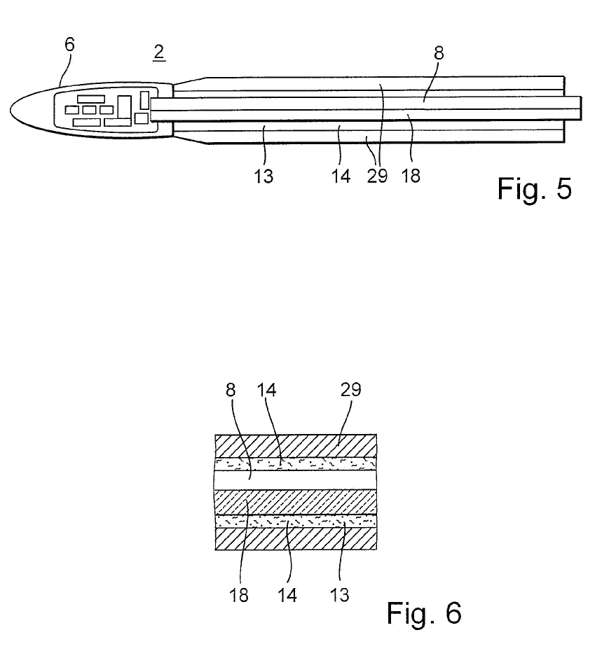

In order to introduce probe head 6 of transdermal measurement probe 2 through the skin into the patient's body, an introducer 28 can be used, for example designed as a canula, tube or metal tube. Particularly suitable are canulas with a longitudinal slit, allowing for easy removal of the introducer 19 after placement of the transdermal measurement probe 2 in the skin. The light conductor 8, 18 can slide out through the open slit in introducer 29. FIG. 5 shows the transdermal measurement probe 2 positioned in the introducer 29. Probe head 6 protrudes from the introducer 29; the light conductor 8, 18 and the sheath structure 14 are stabilized by introducer 29.

A detailed view of parts of the light conductor 8, 18 with the introducer 29 is shown in FIG. 6. The introducer 29 designed as a metal tube encloses the sheath structure 14 and both light conductor 8, 18 to transmit power or data from or to the transdermal measurement probe 2. The tissue-like structure of the canula 13 is clearly visible, providing flexible support, but with high tensile strength, and corresponding protection of the light conductor.

What is claimed is:

1. An analysis device for in vivo determination of an analyte in a patient's body, comprising
a transdermal measurement probe adapted to be introduced through the skin surface into the body, with a probe head including an electro-chemical analysis sensor with two measurement electrodes,
a body-wearable probe connection unit adapted to be worn on the body and for connection to the transdermal measurement probe,
a test circuit connected to the measurement electrodes, wherein after contact of the measurement electrodes with a body fluid, the test circuit generates a test signal characteristic for the desired analytical result,
an evaluation circuit for evaluating test signals from the test circuit and for generating an information about the desired analytical result,
wherein
the test circuit is integrated in the probe head as part of a probe head electronics,
the probe head is coupled to a light conductor,
the probe head includes an optical sensor for converting electrical signals from the probe head electronics into light signals, and for transferring the light signals via the light conductor coupled to the probe head, and
the probe connection unit contains a light receiver for receiving the light signals from the measurement probe for further processing.

2. An analysis device according to claim 1, wherein the test circuit comprises a potentiostat circuit by which the voltage between the measurement electrodes is controlled.

3. An analysis device according to claim 2, wherein the analysis sensor contains an additional reference electrode.

4. An analysis device according to claim 1, wherein the measurement probe comprises a canula which encloses the light conductor at least in the area that is adapted to be transdermally inserted into the body.

5. An analysis device according to claim 4, wherein the canula has a flexible sheath structure with high tensile strength.

6. An analysis device according to claim 5, wherein the flexible sheath structure contains a fibrous fabric.

7. An analysis device according to claim 1, comprising a power supply unit which is adapted to be worn on the body, light from the power supply unit being transported through the light conductor to the measurement probe, the light being converted into electrical energy by an optical receiver disposed in the measurement probe.

8. An analysis device according to claim 7 wherein the power supply unit is integrated in the probe connection unit.

9. An analysis device according to claim 7, wherein the transmission of light energy from and to the measurement probe is carried out by means of light signals which are modulated for data transfer.

10. An analysis device according to claim 1, comprising two light conductors, wherein a first light conductor serves for power transmission to the measurement probe, and a second light conductor serves for transmission of optical data signals.

11. An analysis device according to claim 7, wherein the power supply unit includes a laser diode.

12. An analysis device according to claim 11, wherein the laser diode is a VCSEL laser.

13. An analysis device according to claim 7, wherein the optical receiver in the probe head of the measurement probe is a photodiode.

14. An analysis device according to claim 1, wherein the probe head of the measurement probe comprises an A/D converter for converting the analog electrical signals of the probe head electronics into digital signals which are transmitted from the measurement probe through the light conductor to the probe connection unit.

15. An analysis device according to claim 1, wherein the light conductor consisting of a plastic material.

16. An analysis device according to claim 1, wherein the light conductor have a diameter of no more than 100 μm.

17. An analysis device according to claim 16, wherein the light conductor have a diameter of no more than 30 μm.

18. An analysis device according to claim 17, wherein the light conductor have a diameter of no more than 10 μm.

19. An analysis device according to claim 1, wherein the test circuit is implemented in the probe head as ASIC.

20. An analysis device according to claim 1, wherein the probe head electronics is implemented in the probe head as ASIC.

21. An analysis device according to claim 19, wherein the test circuit is implemented in a silicon technique.

22. An analysis device according to claim 20, wherein the probe head electronics is implemented in the probe head in a silicon technique.

23. An analysis device according to claim 1, wherein the probe head comprising a second electro-chemical analysis sensor.

24. An analysis device according to claim 1, wherein the measurement electrodes are electron-beam sterilized.

25. An analysis device according to claim 24, wherein the electro-chemical analysis sensor is electron-beam sterilized.

26. An analysis device according to claim 24, wherein the probe head of the measurement probe is electron-beam sterilized.

27. An analysis device according to claim 1, wherein the measurement probe comprises a covering unit lying against the skin surface for stabilizing the probe head and/or the light conductor.

28. An analysis device according to claim 18, wherein parts of the evaluation unit are integrated in the covering unit.
